(19) United States
(12) Patent Application Publication
(10) Pub. No.: US 2021/0068628 A1
(43) Pub. Date: Mar. 11, 2021

(54) DISTAL END COVER DEVICE AND ENDOSCOPE

(71) Applicant: OLYMPUS CORPORATION, Tokyo (JP)

(72) Inventor: Koji YAMAYA, Tokyo (JP)

(73) Assignee: OLYMPUS CORPORATION, Tokyo (JP)

(21) Appl. No.: 16/950,175

(22) Filed: Nov. 17, 2020

Related U.S. Application Data
(63) Continuation of application No. PCT/JP2019/007372, filed on Feb. 26, 2019.

(30) Foreign Application Priority Data
May 21, 2018 (JP) 2018-096954

(51) Int. Cl. A61B 1/00; A61B 1/005

(52) U.S. Cl. CPC A61B 1/00137; A61B 1/0057; A61B 1/0052

(57) ABSTRACT

A distal end cover device includes: a distal end cover main body that is formed of a rigid material having insulation properties, and is fixed to an outer periphery of a distal end portion of an insertion portion in a removably attachable manner; a ring cover that is formed of a material having elasticity and insulation properties, and is fixed to outer peripheries of the distal end portion and the distal end cover main body in a removably attachable manner; a raising base provided to the distal end cover main body; a shaft portion that is provided to the distal end cover main body, and couples the raising base to the distal end cover main body in a displaceable manner (swingably); and an operation wire that is coupled to the raising base, and is configured to be pulled by an operator to move the raising base.

### CROSS REFERENCE TO RELATED APPLICATION

This application is a continuation application of PCT/JP2019/007372 filed on Feb. 26, 2019 and claims benefit of Japanese Application No. 2018-096954 filed in Japan on May 21, 2018, the entire contents of which are incorporated herein by this reference.

### BACKGROUND OF THE INVENTION

### 1. Field of the Invention

The present invention relates to a distal end cover device removably attached to an insertion device, such as an endoscope including an insertion portion, and an endoscope including a distal end portion to which a distal end cover is fixed.

### 2. Description of the Related Art

Conventionally, in the medical field, endoscopes have been widely used, which serve as insertion devices that each allow an elongated insertion portion to be inserted into a body cavity, enabling internal organs to be observed from the inside of the body cavity. According to such a type of endoscope, a treatment instrument, which is inserted through a treatment instrument insertion opening provided at an operation portion, is caused to protrude from a distal end portion of the insertion portion, which allows various treatments in body cavities to be applied.

In treatments using such treatment instruments, raising bases (forceps elevators) have been widely used for changing the direction of a distal end of the treatment instrument protruding in a body cavity to a desired direction by an operation on a hand side.

Here, to facilitate cleaning of an endoscope, a configuration where such type of a raising base is provided at a distal end cover that is removably attached to a distal end portion of the endoscope, has been proposed recently. Note that the distal end cover is a member for insulating an outer periphery of the distal end portion, in order to prevent propagation of high-frequency current emitted from the treatment instrument or the like, to an unintentional part. Accordingly, the distal end cover is typically formed of an insulator having elasticity.

For example, Japanese Patent Application Laid-Open Publication No. 2009-273665 discloses a distal end hood (distal end cover device) with a treatment instrument raising device, including: a distal end hood tube (distal end cover) that is formed of an elastic rubber or the like to have a substantially cylindrical shape opening in both forward and backward directions, and is removably attached to a distal end portion main body (distal end portion); the treatment instrument raising base to-and-fro movable in the forward and backward directions in the distal end hood tube; and an operation wire for slidably driving the treatment instrument raising base.

### SUMMARY OF THE INVENTION

A distal end cover device according to an aspect of the present invention includes: a first cover that is formed of a rigid material having insulation properties and is fixed in a removably attachable manner to an outer periphery of a distal end portion of an insertion portion of an endoscope to be inserted into a subject; a second cover that is formed of a material having elasticity and insulation properties and is arranged so as to be in close contact with the distal end portion and the first cover; a movement member configured to be movable; a pulling member that includes one end coupled to the movement member, and another end fixed to a predetermined position of the endoscope; a coupling member that is provided to the first cover, and is configured to couple the movement member to the first cover in a displaceable manner; a movement member operation portion configured to be operated to and fro by an operator to cause the movement member to move; a sheath that is configured to allow the pulling member to be inserted in an inside of the sheath, and includes a proximal end movable in a longitudinal direction of the pulling member with respect to the predetermined position in response to a to-and-fro operation on the movement member operation portion; and a fixation portion that is provided to the first cover, and fixes a distal end of the sheath to the first cover.

An endoscope according to an aspect of the present invention includes the distal end portion to which the distal end cover is fixed.

### BRIEF DESCRIPTION OF THE DRAWINGS

FIG. 1 is a side view showing a configuration of an endoscope apparatus according to a first embodiment of the present invention;

FIG. 2 is a side view showing a state where a distal end cover device is removed from an endoscope according to the first embodiment of the present invention;

FIG. 3 is a perspective view of a distal end cover having not been attached to a distal end portion yet according to the first embodiment of the present invention;

FIG. 4 is a perspective view of the distal end cover attached to the distal end portion according to the first embodiment of the present invention;

FIG. 5 is a sectional view of the distal end portion and the distal end cover according to the first embodiment of the present invention;

FIG. 6 is a sectional view mainly showing a connection portion between a distal end cover main body and a guide sheath according to the first embodiment of the present invention;

FIG. 7 is a plan view of the distal end cover main body according to the first embodiment of the present invention;

FIG. 8 is a perspective view showing a state where the distal end cover is removed from the distal end portion according to the first embodiment of the present invention;

FIG. 9 is a major-part sectional view showing a support structure of the raising base according to the first embodiment of the present invention;

FIG. 10 is a side view showing a configuration of an endoscope apparatus according to a second embodiment of the present invention;

FIG. 11 is a side view showing a state where a distal end cover device is removed from an endoscope according to the second embodiment of the present invention;

FIG. 12 is a perspective view of a distal end cover having not been attached to a distal end portion yet according to the second embodiment of the present invention;

FIG. 13 is a perspective view of the distal end cover attached to the distal end portion according to the second embodiment of the present invention;

FIG. 14 is a sectional view of the distal end portion and the distal end cover according to the second embodiment of the present invention;

FIG. 15 is a side view of the distal end cover device in a case where the raising base is in an inverted state according to the second embodiment of the present invention;

FIG. 16 is a side view of the distal end cover in a case where the raising base is in a raised state according to the second embodiment of the present invention;

FIG. 17 is a sectional view taken along line XVII-XVII of FIG. 15 according to the second embodiment of the present invention;

FIG. 18 is an explanatory diagram showing a state where the raising base is operated while the endoscope is held according to the second embodiment of the present invention;

FIG. 19 is an exploded perspective view showing a major part of a wire mechanism portion according to the second embodiment of the present invention;

FIG. 20 is an explanatory diagram showing margins set to production errors of the distal end cover device according to the second embodiment of the present invention;

FIG. 21 is an explanatory diagram showing margins set to the production errors of the distal end cover device according to the second embodiment of the present invention;

FIG. 22 is a perspective view of the distal end cover attached to the distal end portion according to a modification of the second embodiment of the present invention;

FIG. 23 is an exploded perspective view showing a major part of the wire mechanism portion according to the modification of the second embodiment of the present invention;

FIG. 24 is a perspective view showing a distal end cover having not been attached to a distal end portion yet according to a third embodiment of the present invention;

FIG. 25 is a perspective view of the distal end cover attached to the distal end portion according to the third embodiment of the present invention; and

FIG. 26 is a perspective view of a distal end cover having not been attached to a distal end portion yet according to a modification of the third embodiment of the present invention.

### DETAILED DESCRIPTION OF THE PREFERRED EMBODIMENTS

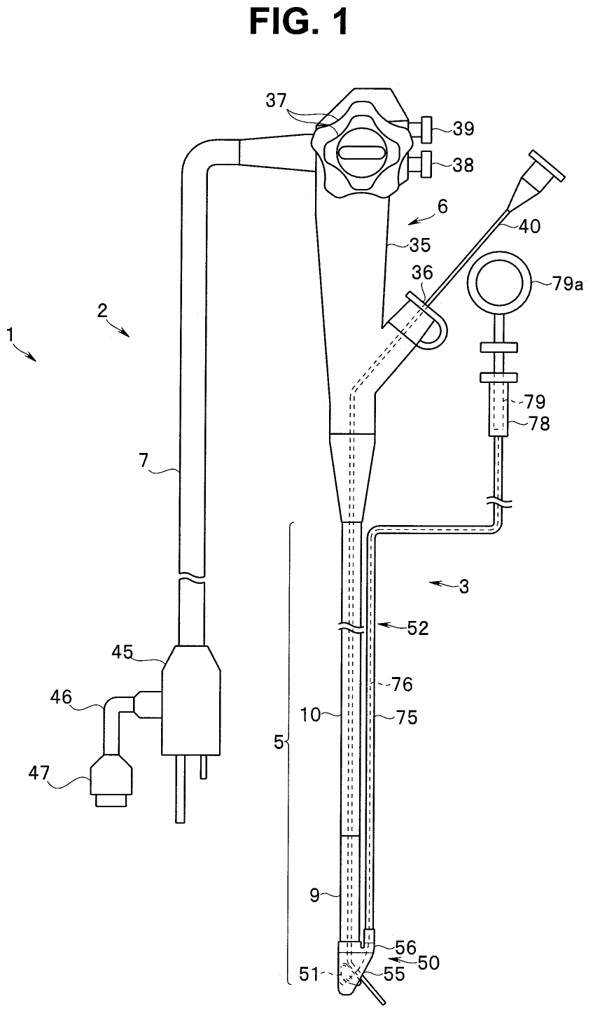

Hereinafter, referring to FIGS. 1 to 9, a first embodiment of the present invention is described.

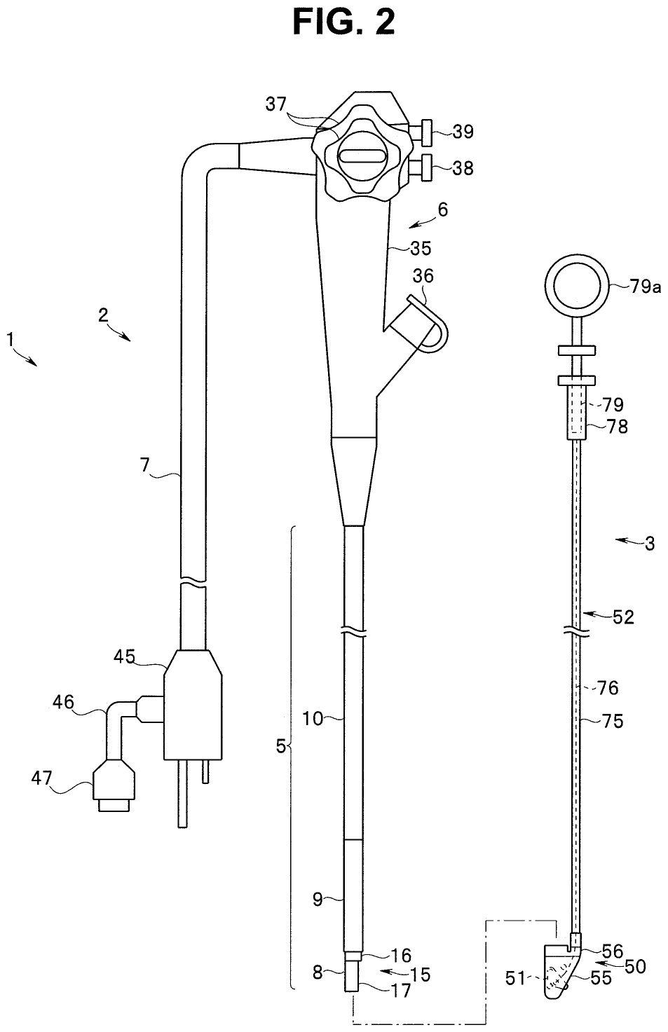

As shown in FIGS. 1 and 2, an endoscope apparatus 1 of the present embodiment includes: an endoscope 2; and a distal end cover device 3 removably attached to the endoscope 2.

The endoscope 2 includes an insertion portion 5, an operation portion 6, and a universal cable 7. The insertion portion 5 is an elongated long member, and has a configuration where a distal end portion 8, a bending portion 9, and a flexible tube portion 10 are consecutively provided from the distal end side.

The distal end portion 8 includes a distal end configuration part 15 formed of metal, such as stainless steel. The distal end configuration part 15 includes a base portion 16, and an observation protrusion portion 17 that protrudes from a distal end of the base portion 16 in an insertion axis O direction. A ring-shaped insulation member 20 having insulation properties is provided on the outer periphery of the distal end configuration part 15 on a proximal end side. Furthermore, at a position nearer to the proximal end than the insulation member 20, an envelope 21 having a distal end fixed to the distal end configuration part 15 with a thread-wound bonding portion 21a is consecutively provided.

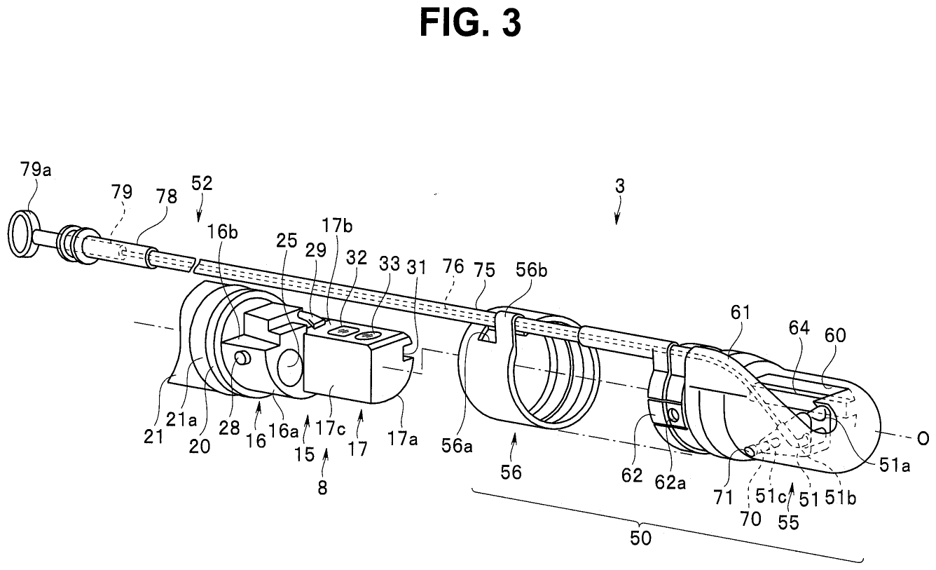

For example, as shown in FIG. 3, a schematic shape of the base portion 16 is formed to be a shape obtained by cutting off substantially half a cylinder (i.e., substantially a half-cylindrical shape). Accordingly, the base portion 16 includes a circular arc surface portion 16a and a planar surface portion 16b on the outer periphery.

A schematic shape of the observation protrusion portion 17 is formed to be a shape obtained by cutting off substantially three quarters of a cylinder arranged coaxially with the base portion 16 (i.e., substantially a one-quarter cylindrical shape). Accordingly, the observation protrusion portion 17 includes, on the outer periphery: a circular arc surface portion 17a; a first planar surface portion 17b, for example, parallel to the planar surface portion 16b of the base portion 16; and a second planar surface portion 17c, for example, orthogonal to the first planar surface portion 17b.

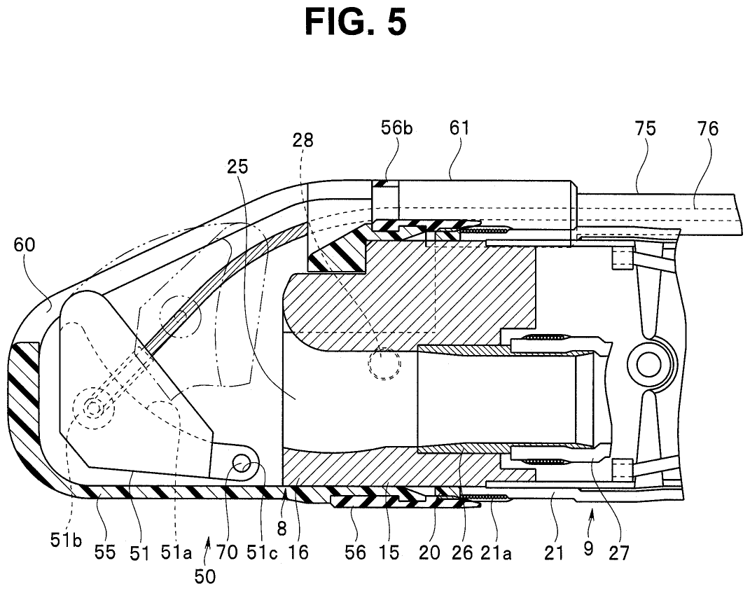

A treatment instrument protrusion port 25 is provided, on an end surface of the base portion 16, at a position adjacent to the second planar surface portion 17c of the observation protrusion portion 17. As shown in FIG. 5, on a proximal end side of the distal end configuration part 15, a coupling pipe 26 communicates with the treatment instrument protrusion port 25, and furthermore, a distal end side of a treatment instrument channel 27 inserted into the insertion portion 5 communicates with the coupling pipe 26.

On the circular arc surface portion 16a of the base portion 16, an engagement pin 28 protruding in a direction orthogonal to the insertion axis O is provided.

A nozzle 29 for air feeding or water feeding toward the first planar surface portion 17b formed on the observation protrusion portion 17 is provided on the base portion 16 in a protruding manner.

A key groove 31 extending along the insertion axis O is provided in the circular arc surface portion 17a of the observation protrusion portion 17.

The first planar surface portion 17b of the observation protrusion portion 17 is provided with an observation window 32 of an image pickup unit, not shown, and an illumination window 33 of an illumination unit, not shown. Such arrangement of the observation window 32 allows the endoscope 2 of the present embodiment to be what is called a side-viewing endoscope that has an observation field of view in a direction intersecting with the insertion axis O.

The bending portion 9 is configured to be bendable in four directions (upward, downward, left and right directions) orthogonal to the insertion axis O, for example. The flexible tube portion 10 is a tubular member that is long and has flexibility. Note that the upward and downward directions and the left and right directions described here are defined with respect to the insertion portion 5 for the sake of convenience, and for example, each of the directions is defined with reference to the direction where the observation window 32 is provided (upward direction).

The operation portion 6 is consecutively provided at a proximal end of the flexible tube portion 10. At the middle of the operation portion 6, a grasping portion 35 configured to be grasped by an operator or the like is configured.

On a distal end side of the grasping portion 35, the operation portion 6 is provided with a treatment instrument insertion opening 36. The treatment instrument insertion opening 36 communicates with a proximal end side of the treatment instrument channel 27, in the operation portion 6. Accordingly, for example, as shown in FIG. 1, a treatment instrument 40 serving as another insertion device inserted into the treatment instrument insertion opening 36 is allowed to protrude from the treatment instrument protrusion port 25 of the distal end portion 8.

On a proximal end side of the grasping portion 35, the operation portion 6 is provided with, for example, an angle knob 37 for allowing the bending portion 9 to be subjected to a bending operation, an air/water feeding button 38 for allowing air feeding or water feeding through the nozzle 29 provided at the distal end portion 8, and a suction button 39 for sucking a suction target object in a subject through the treatment instrument protrusion port 25.

The universal cable 7 extends from a side surface of the operation portion 6 on the proximal end side. At an end portion of the universal cable 7, an endoscope connector 45 to be connected to a light source device (not shown) is provided. From a side of the endoscope connector 45, a signal transmission cable 46 extends. On the other end side of the signal transmission cable 46, an electric connector 47 to be connected to a video processor (not shown) is provided.

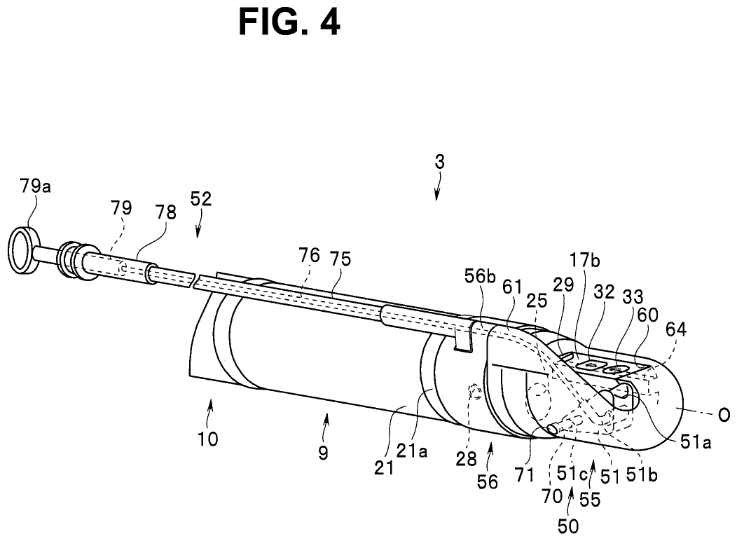

As shown in FIGS. 3 and 4, the distal end cover device 3 includes: a distal end cover 50 removably attached to the distal end portion 8; a raising base 51 that is provided in the distal end cover 50 and serves as an operation member; and a wire mechanism portion 52 for operation of the raising base 51 from the hand side.

The distal end cover 50 includes: a distal end cover main body 55 serving as a first cover; and a ring cover 56 serving as a second cover.

The distal end cover main body 55 is formed of, a material, such as plastic, which is rigid and has insulation properties. The distal end cover main body 55 has a substantially cylindrical shape with a hemispherically closed distal end, and can be fixed to the outer periphery of the distal end portion 8 in a removably attachable manner mainly so as to cover the distal end configuration part 15.

The distal end cover main body 55 is provided with an opening portion 60 through which the observation window 32 and the illumination window 33 are exposed and which is for allowing the treatment instrument 40 protruding from the treatment instrument protrusion port 25 to be led out.

A sheath connection portion 61 serving as a fixation portion to which a distal end of a guide sheath 75 constituting the after-mentioned wire mechanism portion 52 is to be connected is provided to protrude from one side of the opening portion 60 in the distal end cover main body 55.

At the distal end cover main body 55, an engagement piece 62 is formed on one side of the opening portion 60 and the sheath connection portion 61 and on a proximal end side of the distal end cover main body 55. The engagement piece 62 can be elastically deformed in the outer diameter direction of the distal end cover main body 55. The engagement piece 62 is provided with an engagement hole 62a that serves as an engagement portion engageable with the engagement pin 28 protruding from the base portion 16 of the distal end configuration part 15.

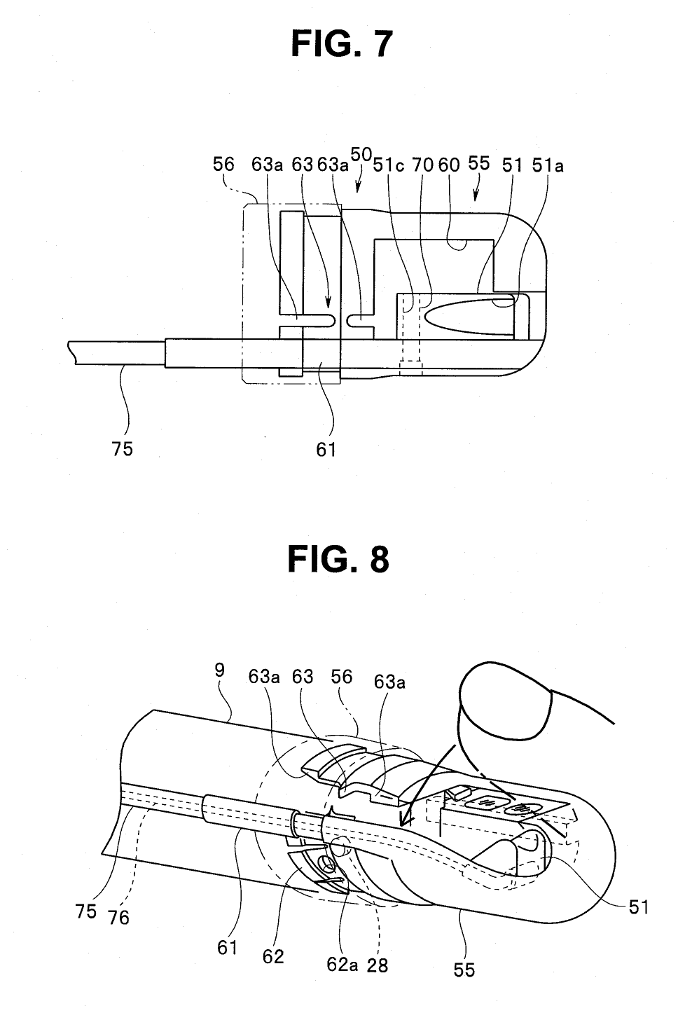

As shown in FIG. 7, the distal end cover main body 55 is provided with a slit 63a on the other side of the sheath connection portion 61 and on a proximal end side of the opening portion 60. With the slit 63a, at the distal end cover main body 55, a fragile portion 63 allowing the distal end cover main body 55 to be broken in an expanding direction is configured.

Such arrangement of the fragile portion 63 on the other side of the sheath connection portion 61 allows the sheath connection portion 61 to be arranged between the engagement hole 62a and the fragile portion 63 in a circumferential direction of the distal end cover main body 55.

Furthermore, in the distal end cover main body 55, a key 64 to be fit with the key groove 31 is provided. Fitting of the key 64 with the key groove 31 enables the distal end cover main body 55 to be positioned at an appropriate position with respect to the distal end configuration part 15.

The ring cover 56 is formed of, for example, a material, such as silicone rubber, having elasticity and insulation properties, into a ring-shaped member having a diameter smaller than the outer diameters of the insulation member 20 of the distal end portion 8 and the distal end cover main body 55.

The ring cover 56 is elastically deformed in a diameter-expanding direction, and thus can be arranged at a position to integrally cover a proximal end region of the distal end portion 8 provided with the insulation member 20 and a proximal end region of the distal end cover main body 55. The ring cover 56 can be brought into close contact with the outer peripheries of the distal end portion 8 (insulation member 20) and the distal end cover main body 55 over the entire peripheries owing to a restoring force by the elasticity.

Note that the ring cover 56 of the present embodiment is provided with a hole portion 56a and a bridge portion 56b for preventing interference with the sheath connection portion 61.

For example, as shown in FIG. 5, the raising base 51 is made of a metal member having a substantially triangular shape in a side view.

On an upper surface of the raising base 51, a guide groove 51a is provided that is inclined from the proximal end toward the distal end at a predetermined elevation angle.

A wire connection portion 51b for connection with an after-mentioned operation wire 76 of the wire mechanism portion 52 is provided at a side portion of the raising base 51 on the distal end side.

A proximal end portion of the raising base 51 is provided with a shaft insertion hole 51c. A shaft portion 70 serving as a coupling member is inserted into the shaft insertion hole 51c. The shaft portion 70 is supported by the distal end cover main body 55. Accordingly, the raising base 51 is coupled with the distal end cover main body 55 in a swingable manner (a displaceable manner).

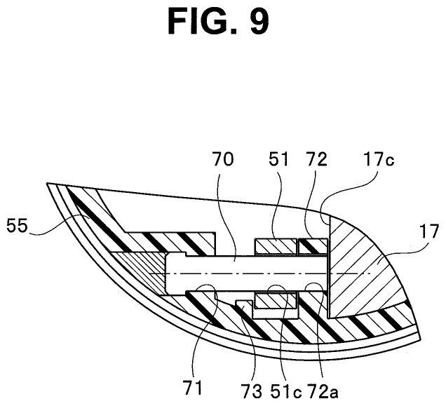

More specifically, for example, as shown in FIG. 9, a first bearing hole 71 communicating with the inside and outside of the distal end cover main body 55 is provided on one side of the distal end cover main body 55.

A bearing protrusion 72 is provided in the distal end cover main body 55. The bearing protrusion 72 is provided with a second bearing hole 72a coaxial with the first bearing hole 71.

The shaft portion 70 is supported by the first and second bearing holes 71 and 72a. Accordingly, the raising base 51 is swingably supported in the distal end cover main body 55.

In the distal end cover main body 55, a restriction protrusion 73 is provided that is for restricting movement of the raising base 51 in the shaft center direction of the shaft portion 70 by causing the proximal end side of the raising base 51 to intervene with the bearing protrusion 72.

Here, the bearing protrusion 72 and the restriction protrusion 73 are provided at positions biased to one side from the observation protrusion portion 17 of the distal end configuration part 15 when the distal end cover main body 55 is attached to the distal end portion 8.

Accordingly, when the distal end cover main body 55 is attached to the distal end portion 8, the raising base 51 is arranged in a space formed between an inner wall of the distal end cover main body 55 and the second planar surface portion 17c of the observation protrusion portion 17, and the guide groove 51a of the raising base 51 faces the treatment instrument protrusion port 25.

For example, as shown in FIGS. 3 and 4, the wire mechanism portion 52 includes the guide sheath 75, and the operation wire 76 serving as a pulling member inserted into the guide sheath 75.

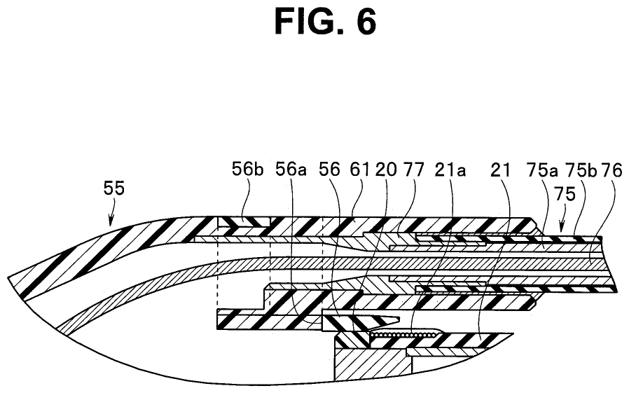

As shown in FIG. 6, the guide sheath 75 includes a coil 75a having flexibility, and an envelope 75b covering an outer periphery of the coil 75a.

A distal end pipe sleeve 77 is connected to the distal end of the guide sheath 75. By fitting of the distal end pipe sleeve 77 with the sheath connection portion 61, the guide sheath 75 is connected to the distal end cover main body 55.

Accordingly, the operation wire 76 inserted into the guide sheath 75 is introduced into the distal end cover main body 55. A distal end of the operation wire 76 is connected to the wire connection portion 51b provided to the raising base 51. The wire connection portion 51b is rotatable with respect to the raising base 51.

On the other hand, a cylinder portion 78 that supports a wire shaft 79 in a to-and-fro movable manner is connected to a proximal end of the guide sheath 75. A proximal end of the operation wire 76 is connected to a distal end of the wire shaft 79. A raising base operation member 79a that has a ring shape and serves as a movement member operation portion, for example, is consecutively provided on the proximal end side of the wire shaft 79.

By moving the wire shaft 79 to and fro through an operation to the raising base operation member 79a, the raising base 51 can be displaced between a raised position and an inverted position.

According to such an embodiment, a distal end cover device 3 includes: a distal end cover main body 55 that is formed of a rigid material having insulation properties, and is fixed to an outer periphery of a distal end portion 8 of an insertion portion 5 in a removably attachable manner; a ring cover 56 that is formed of a material having elasticity and insulation properties, and is fixed to the distal end portion 8 and the distal end cover main body 55 in a removably attachable manner; a raising base 51 provided to the distal end cover main body 55; a shaft portion 70 that is provided to the distal end cover main body 55, and couples the raising base 51 to the distal end cover main body 55 in a displaceable manner (swingably); and an operation wire 76 that is coupled to the raising base 51, and is configured to be pulled by an operator to move the raising base 51. Accordingly, while the insulation properties of the distal end portion 8 are secured, the raising base 51 can be appropriately moved.

In other words, the distal end cover main body 55 fixed to the outer periphery of the distal end portion 8 of the insertion portion 5 in a removably attachable manner is formed of a rigid material having insulation properties, and the raising base 51 is coupled swingably to the distal end cover main body 55 through the shaft portion 70. Accordingly, a fine operation on the hand side to the operation wire 76 can be appropriately transmitted to the raising base 51. In this case, the ring cover 56 formed of a material having elasticity and insulation properties is fixed to the distal end portion 8 and the distal end cover main body 55 in a removably attachable manner. Accordingly, even in a case where the distal end cover main body 55 is made of a rigid material, a joining portion of the distal end portion 8 and the distal end cover main body 55 can be insulated by the ring cover 56 without any gap.

In this case, particularly, the ring cover 56 is in close contact over the entire periphery with, for example, the insulation member 20 of the distal end portion 8 and the proximal end side of the distal end cover main body 55 owing to the elasticity. Accordingly, high-frequency current emitted from the treatment instrument or the like can be appropriately prevented from propagating to an unintentional part.

The operation wire 76 is connected to the distal end cover 50 (distal end cover main body 55) via the guide sheath 75. Accordingly, there is no need to provide the endoscope 2 with a drive shaft rotated by the operation wire 76 and a seal member for sealing the drive shaft, the structure of the endo scope 2 can be simplified, and the endoscope 2 can be effectively cleaned.

The base portion 16 of the distal end configuration part 15, which constitutes the distal end portion 8, is provided with the engagement pin 28, and the engagement hole 62a provided in the distal end cover main body 55 is caused to be engaged with the engagement pin 28. Accordingly, by a simple operation, the distal end cover main body 55 can be fixed to the distal end portion 8.

In this case, the distal end cover main body 55 is provided with the fragile portion 63, and the fragile portion 63 is broken, thereby enabling the used distal end cover main body 55 to be easily separated from the distal end portion 8.

Furthermore, the sheath connection portion 61 protruding in the outer diameter direction from the distal end cover main body 55 is arranged between the engagement hole 62a and the fragile portion 63. Accordingly, for example, as shown in FIG. 8, by a simple operation of pushing the sheath connection portion 61 toward the engagement hole 62a, the distal end cover main body 55 can be broken in a direction where engagement of the engagement hole 62a with the engagement pin 28 is released.

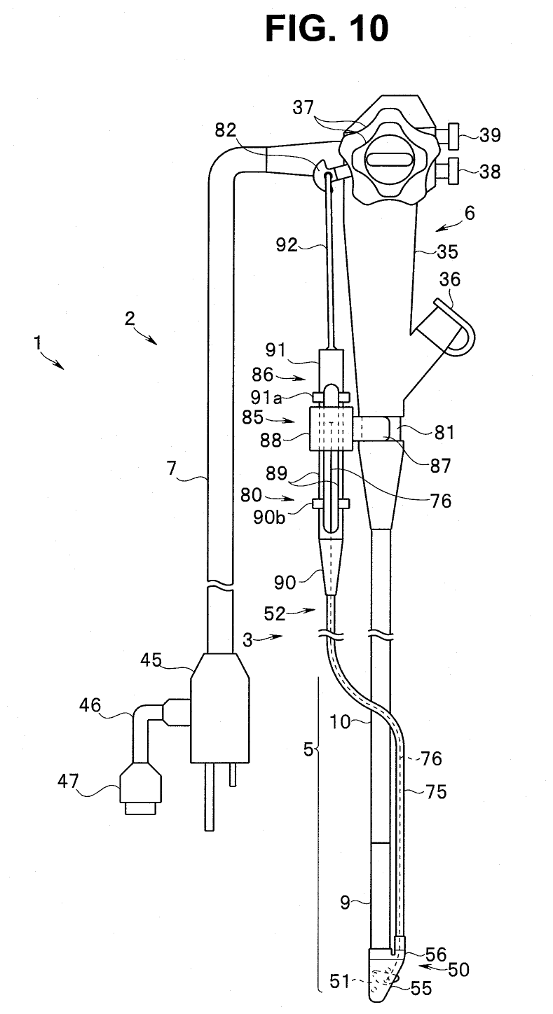

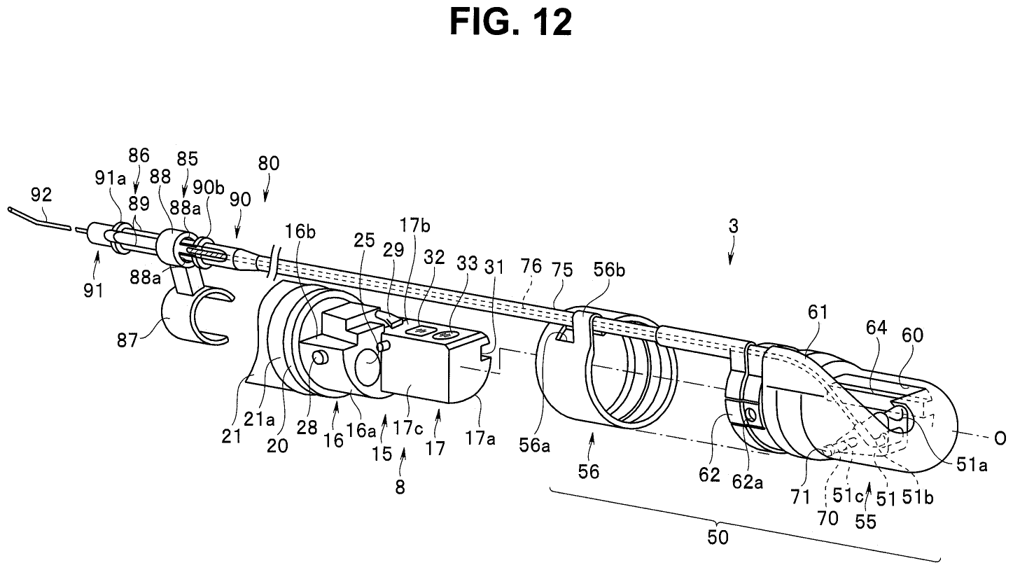

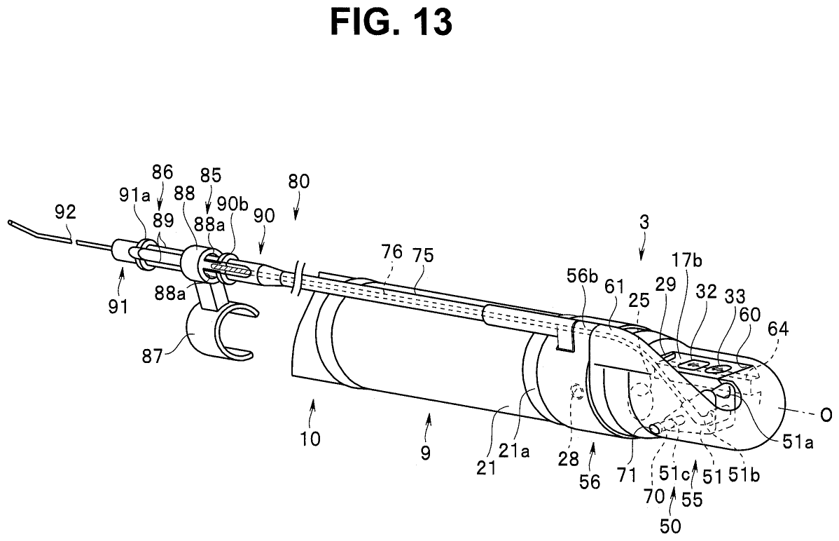

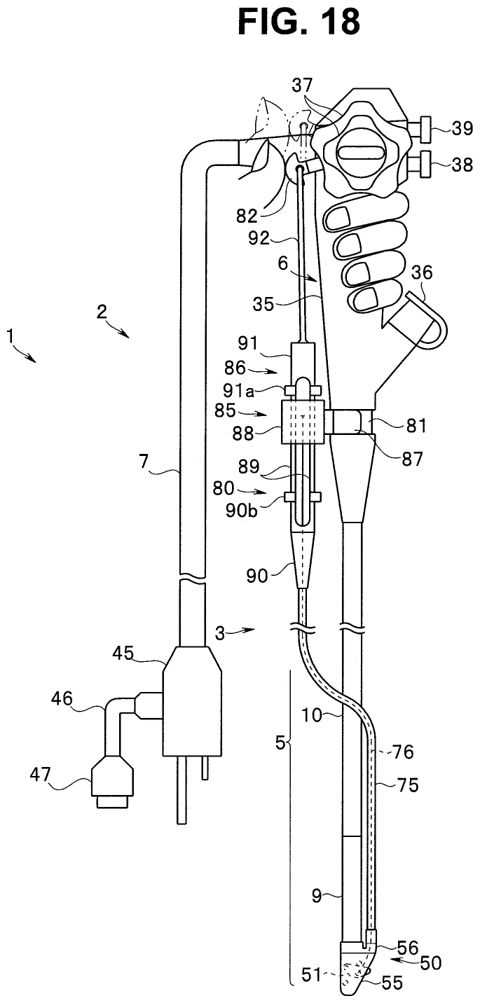

Next, referring to FIGS. 10 to 21, a second embodiment of the present invention is described.

Note that the present embodiment is different mainly in the configuration of the wire mechanism portion of the distal end cover device from the first embodiment described above. Furthermore, components equivalent to the components of the first embodiment described above are appropriately assigned the same symbols, and the description is omitted.

The present embodiment fixes a wire mechanism portion 80 to the operation portion 6 of the endoscope 2, and enables operations to the raising base 51 and various operations to the endoscope 2 to be integrally performed.

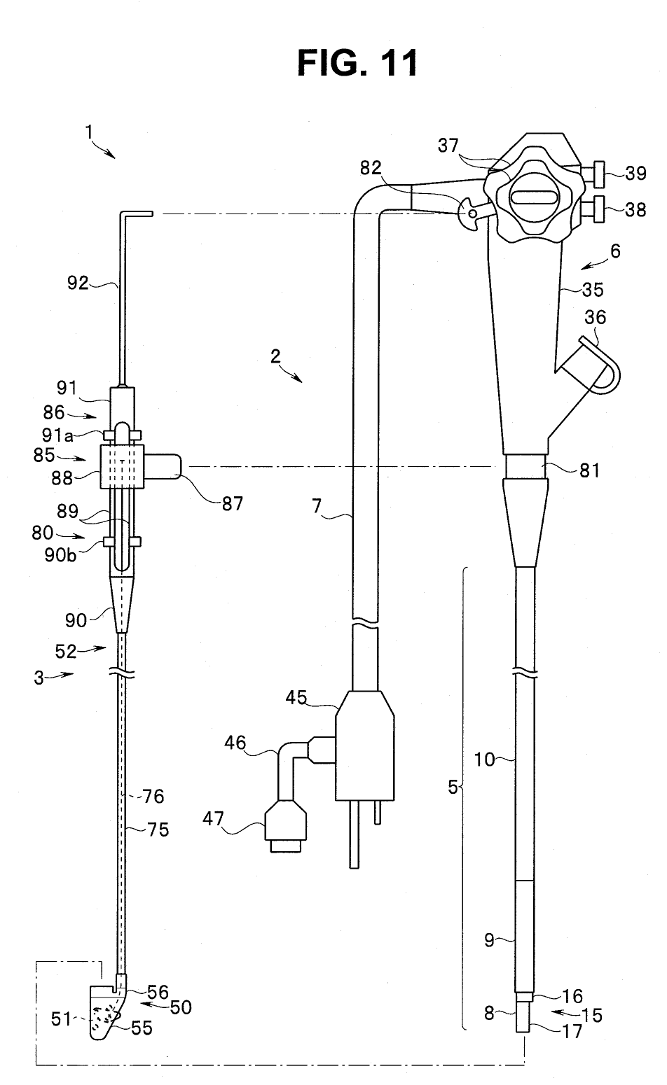

Accordingly, for example, as shown in FIGS. 10 and 11, the periphery of the operation portion 6 of the endoscope 2 in the present embodiment is provided with a fixation groove 81 for fixing the wire mechanism portion 80. Furthermore, the operation portion 6 of the endoscope 2 is provided with a raising base operation knob 82 swingable coaxially with the angle knob 37.

As shown in FIGS. 10 to 13, the wire mechanism portion 80 in the present embodiment includes a fixation member 85 that fixes the proximal end of the operation wire 76 to the operation portion 6, and a slider 86 movable relatively to the fixation member 85.

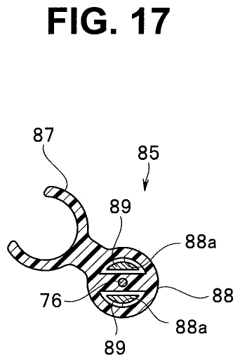

As shown in FIG. 17, the fixation member 85 includes a C-shape clip 87 removably attached to the fixation groove 81, and a fixation block 88 formed integrally with the C-shape clip 87.

The fixation block 88 is provided with a pair of slide holes 88a. The proximal end of the operation wire 76 is fixed to a part intervening between these slide holes 88a.

The slider 86 includes: a pair of slide bars 89 inserted into the respective slide holes 88a of the fixation block 88; a slider distal end portion 90 that couples the distal end sides of the slide bars 89; and a slider proximal end portion 91 that couples the proximal end sides of the slide bars 89.

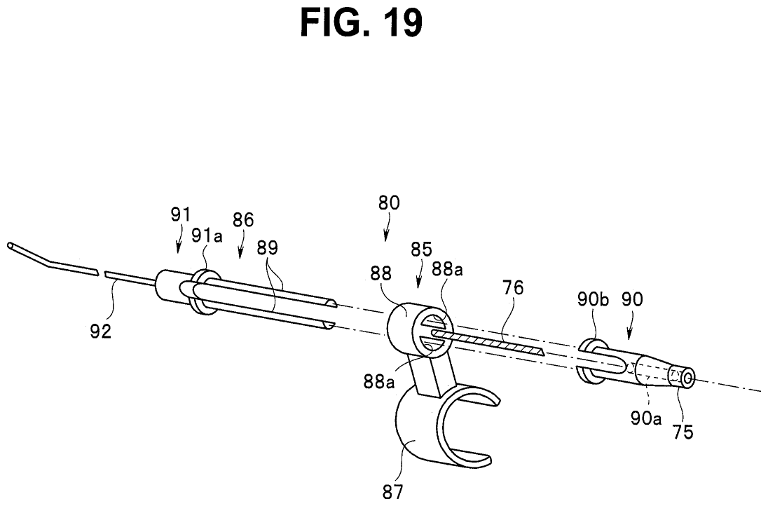

The proximal end of the guide sheath 75 is connected to the slider distal end portion 90. The slider distal end portion 90 is provided with an insertion hole 90a that allows the operation wire 76 fixed to the fixation block 88 to be inserted into the guide sheath 75 (see FIG. 19).

Furthermore, the distal end sides of the slide bars 89 are provided with a flange portion 90b that restricts the relative movement of the slider 86 toward the proximal end with respect to the fixation block 88.

The slider proximal end portion 91 is provided with a coupling bar 92 that couples the slider proximal end portion 91 to the raising base operation knob 82.

Furthermore, the proximal end sides of the slide bars 89 are provided with a flange portion 91a that restricts the relative movement of the slider 86 toward the distal end with respect to the fixation block 88.

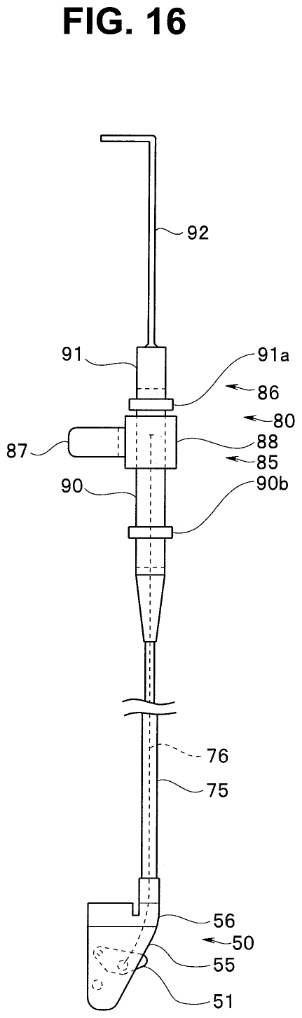

According to such a configuration, when the operator inclines the raising base operation knob 82 toward the distal end of the operation portion 6, the amount of protrusion of the slider 86 on the distal end side of the fixation member 88 increases (see FIGS. 10 and 16). Accordingly, a substantially effective length of the guide sheath 75 (i.e., a total value of the length of the guide sheath 75 itself and the amount of protrusion of the slider 86 protruding toward the distal end of the fixation member 88) between the fixation member 88 and the sheath connection portion 61 increases (see FIG. 10).

As a result, the operation wire 76 is pulled with respect to the guide sheath 75, and the raising base 51 is raised (stands up).

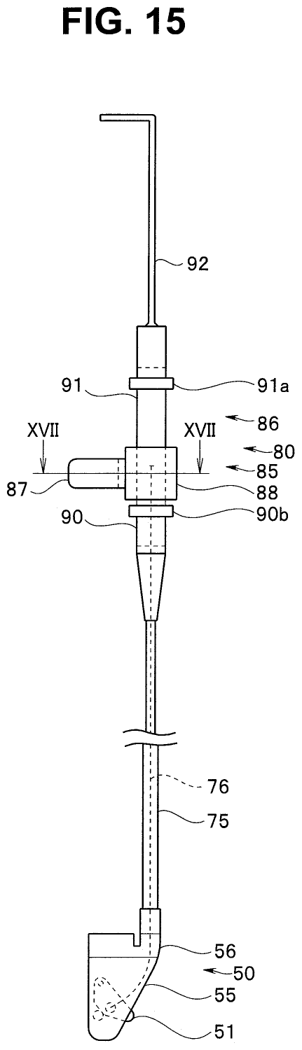

On the other hand, when the operator inclines the raising base operation knob 82 toward the proximal end of the operation portion 6, the amount of protrusion of the slider 86 on the distal end side of the fixation member 88 decreases (see FIG. 15). Accordingly, the substantially effective length of the guide sheath 75 (i.e., the total value of the length of the guide sheath 75 itself and the amount of protrusion of the slider 86 protruding toward the distal end of the fixation member 88) between the fixation member 88 and the sheath connection portion 61 decreases.

As a result, the operation wire 76 is pushed out of the guide sheath 75, and the raising base 51 is inverted (taken down).

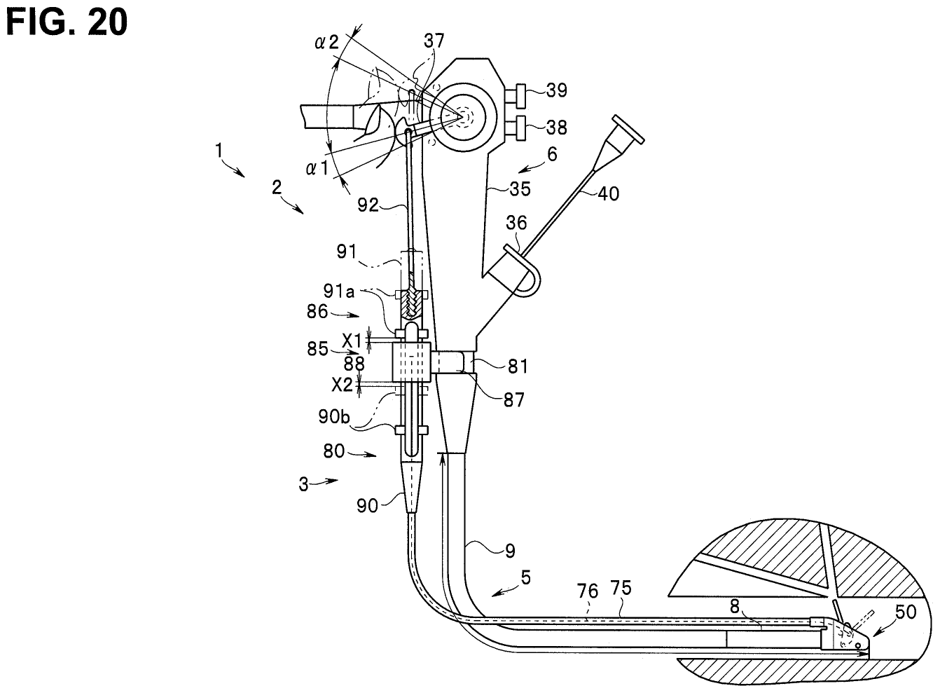

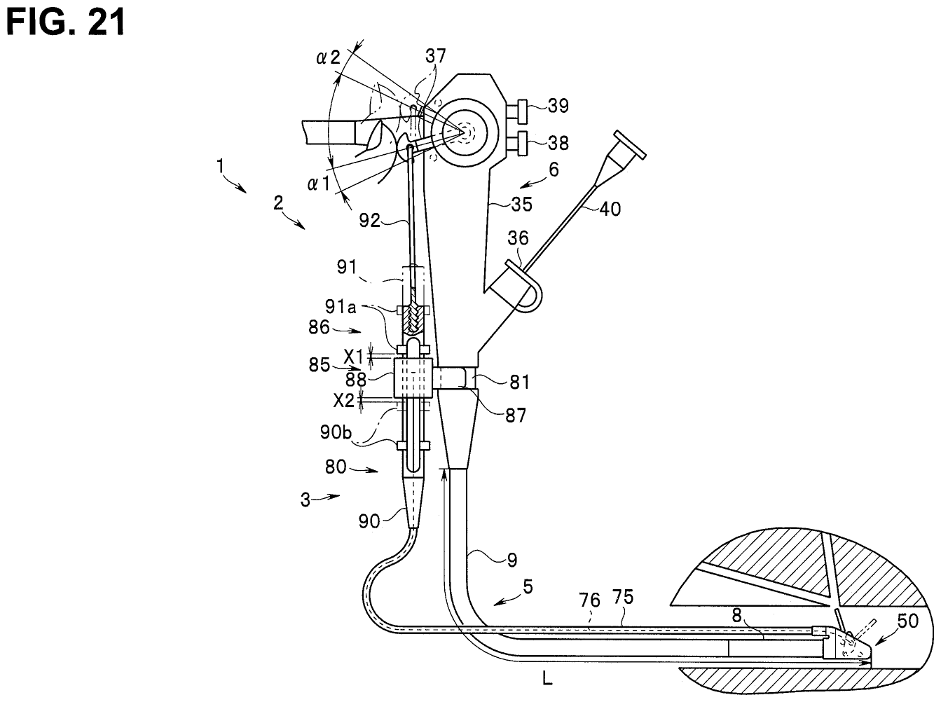

In such a wire mechanism portion 80, to improve the yield against production errors, such as of the lengths of the guide sheath 75, the operation wire 76 and the coupling bar 92, for example, as shown in FIGS. 20 and 21, it is preferable to provide predetermined margins x1 and x2 at an advanced position and a retracted position of the slider 86 that are defined by the respective flange portions 90b and 91a. Note that if such margins x1 and x2 are provided, predetermined margins α1 and α2 are also provided for swing angles allowed for the raising base operation knob 82.

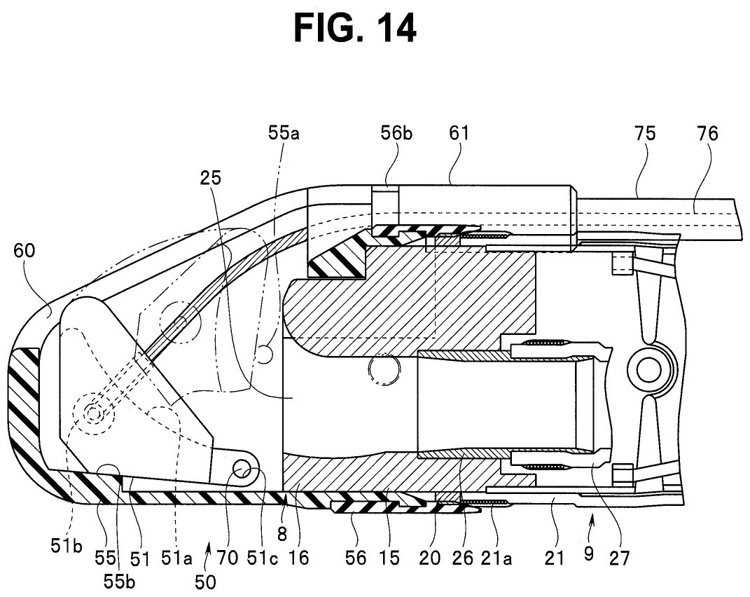

To prevent the raising base 51 from excessively raised or inverted owing to providing of such margins, for example, as shown in FIG. 14, it is preferable to form stoppers 55a and 55b that define the raised position and the inverted position for the distal end cover main body 55.

Note that for example, as shown in FIGS. 20 and 21, also in the endoscope 2, production errors, such as of the length L of the insertion portion 5, can occur. Such production errors can be resolved by deflections of the guide sheath 75 and the operation wire 76 with respect to the insertion portion 5 or the like.

According to such an embodiment, in addition to the working effects obtained by the first embodiment described above, an advantageous effect is exerted that can perform the operation to the raising base 51 integrally with the operation to the endoscope 2.

In this case, the sheath connection portion 61 serving as a fixation portion is provided to the distal end cover main body 55, and the substantially effective length of the guide sheath 75 is increased and reduced with reference to the sheath connection portion 61. Accordingly, even when the proximal end of the operation wire 76 is fixed to the operation portion 6 by the fixation member 85, the raising base 51 can be appropriately moved.

In particular, in the present embodiment, the configuration is made such that the proximal end of the operation wire 76 is fixed to the operation portion 6, and when the raising base operation knob 82 is inclined toward the distal end of the operation portion 6, the substantially effective length of the guide sheath 75 is increased to raise the raising base 51. Accordingly, when the raising base operation knob 82 is inclined toward the distal end of the operation portion 6, the raising base 51 can be raised, and the operation direction and the movement of the raising base 51 can be allowed to conform with feeling of the operator.

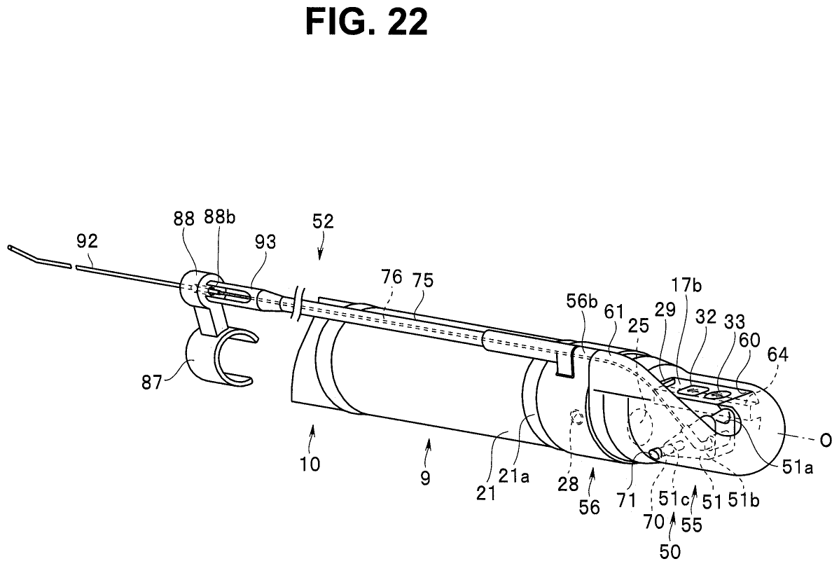

Here, in the second embodiment described above, the configuration is described where by extending and contracting the guide sheath 75, the operation wire 76 is resultantly allowed to move to-and-fro with respect to the distal end cover main body 55. For example, as shown in FIG. 22, a configuration of directly moving the operation wire 76 to-and-fro with respect to the guide sheath 75 can be adopted.

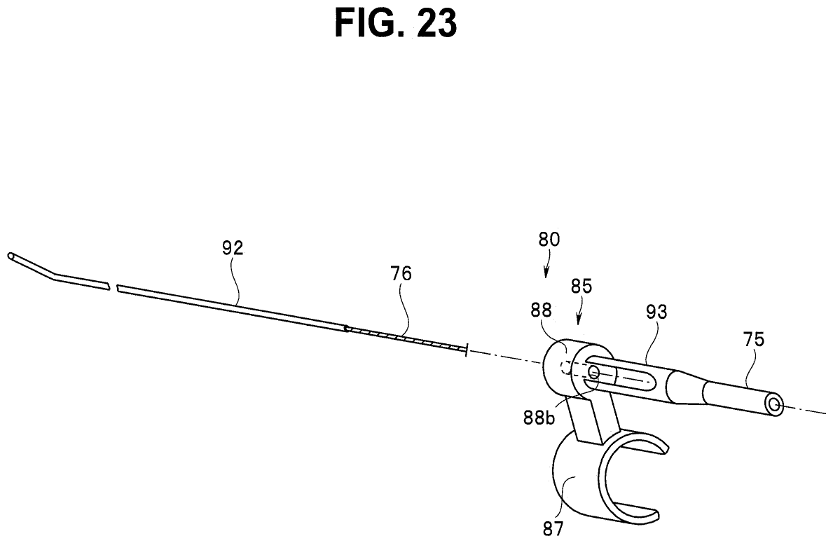

In this case, for example, as shown in FIG. 23, the proximal end of the guide sheath 75 is fixed to the fixation block 88 via a coupler 93.

The proximal end of the operation wire 76 passes through the wire insertion hole 88b provided to the fixation block 88, and is subsequently connected to the coupling bar 92.

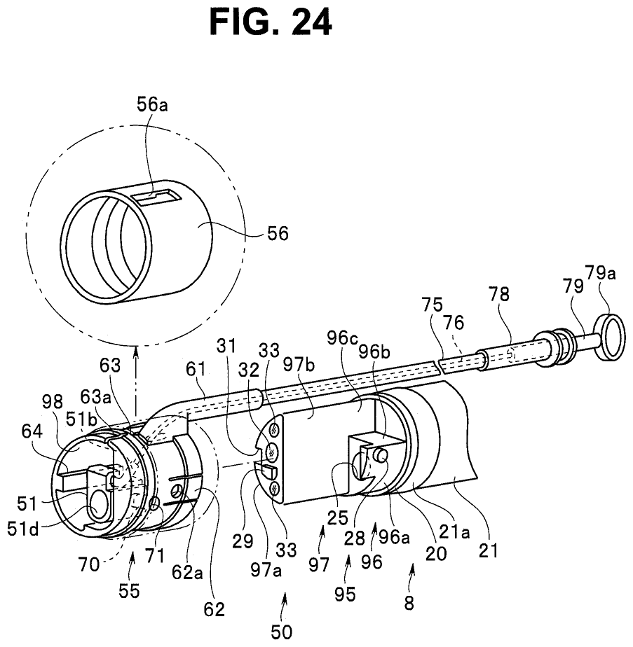

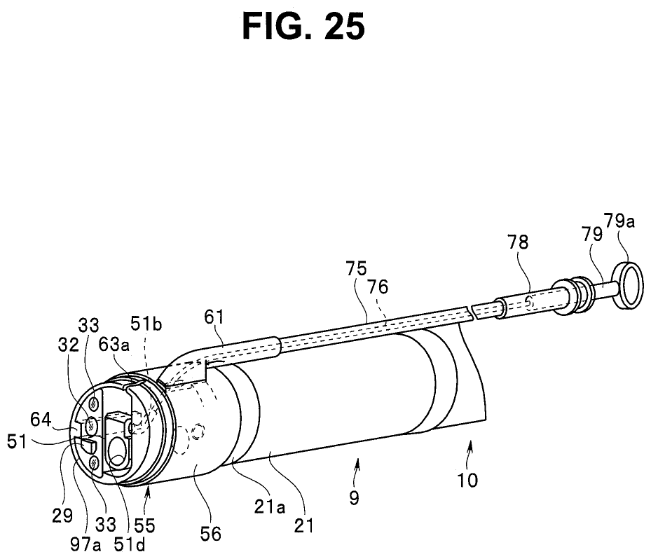

Next, FIGS. 24 to 26 relate to a third embodiment of the present invention. FIG. 24 is a perspective view showing the distal end cover having not been attached to the distal end portion yet. FIG. 25 is a perspective view showing the distal end cover attached to the distal end portion. Note that while the aforementioned first embodiment describes the configuration of applying the distal end cover device 3 to what is called a side-viewing endoscope 2, the present embodiment is different mainly in that the distal end cover is applied to what is called a direct-viewing endoscope 2 having a field of view in the insertion axis O direction. Furthermore, components equivalent to the components of the first embodiment described above are appropriately assigned the same symbols, and the description is omitted.

As shown in FIG. 24, the distal end configuration part 95 of the present embodiment includes a base portion 96, and an observation protrusion portion 97 that protrudes from a distal end of the base portion 96 in an insertion axis O direction.

For example, as shown in FIG. 24, a schematic shape of the base portion 96 is formed to be a shape obtained by cutting off substantially a quarter of a cylinder. Accordingly, the base portion 96 includes, on the outer periphery: a circular arc surface portion 96a; a first planar surface portion 96b; and a second planar surface portion 96c that intersects with the first planar surface portion 96b.

A schematic shape of the observation protrusion portion 97 is formed to be a shape obtained by cutting off substantially half a cylinder arranged coaxially with the base portion 96 (i.e., substantially a half-cylindrical shape). Accordingly, the observation protrusion portion 97 includes, on the outer periphery: a circular arc surface portion 97a; and a planar surface portion 97b that is in parallel to the second planar surface portion 96c of the base portion 96.

A treatment instrument protrusion port 25 is provided, on an end surface of the base portion 96, at a position adjacent to the planar surface portion 97b of the observation protrusion portion 97.

The end surface of the observation protrusion portion 97 is provided with an observation window 32 of an image pickup unit, not shown, an illumination window 33 of an illumination unit, not shown, and a nozzle 29 for air feeding or water feeding toward the observation window 32. Such arrangement of the observation window 32 allows the endoscope 2 of the present embodiment to be what is called a direct-viewing endoscope that has an observation field of view in the insertion axis O direction.

A distal end cover device 3 having a configuration substantially the same as the configuration shown in the first embodiment described above can be applied also to the distal end portion 8 of the endoscope 2 where the distal end configuration part 95 is configured as described above. Note that as shown in FIGS. 24 and 25, an opening portion 98 is provided to the distal end cover main body 55 in the present embodiment, not at the side portion of the distal end cover main body 55 but at the distal end.

The raising base 51 in the present embodiment is provided with a guide hole 51d instead of the guide groove 51a.

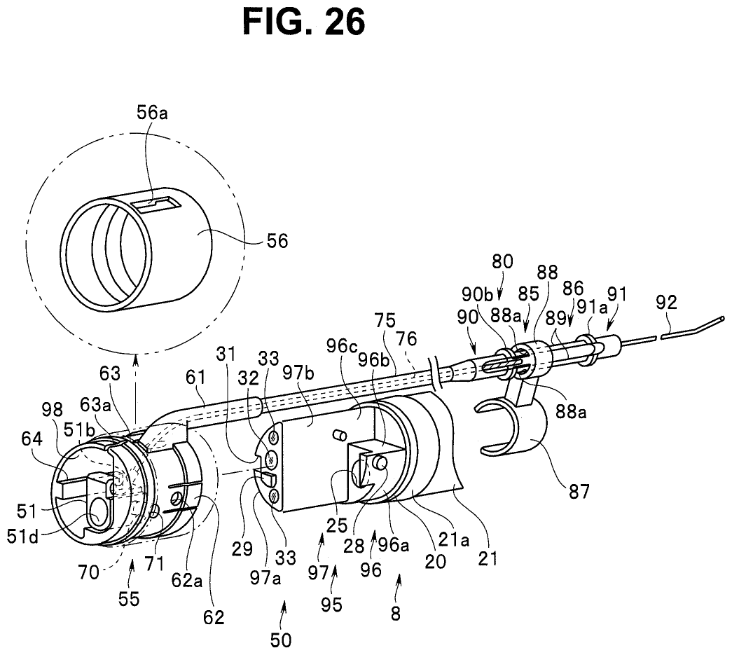

Here, to the distal end cover device 3 in such an embodiment, for example, as shown in FIG. 26, the configuration of the wire mechanism portion 80 and the like shown in the second embodiment described above can be applied. The configuration of the wire mechanism portion 80 shown in FIG. 23 is also applicable.

Note that the present invention is not limited to each embodiment and each modification described above and can be variously changed and modified, and such change and modification are also encompassed in the technical scope of the present invention.

What is claimed is:

1. A distal end cover device, comprising:
a first cover that is formed of a rigid material having insulation properties and is fixed in a removably attachable manner to an outer periphery of a distal end portion of an insertion portion of an endoscope to be inserted into a subject;
a second cover that is formed of a material having elasticity and insulation properties and is arranged so as to be in close contact with the distal end portion and the first cover;
a movement member configured to be movable;
a pulling member that includes one end coupled to the movement member, and another end fixed to a predetermined position of the endoscope;
a coupling member that is provided to the first cover, and is configured to couple the movement member to the first cover in a displaceable manner;
a movement member operation portion configured to be operated to and fro by an operator to cause the movement member to move;
a sheath that is configured to allow the pulling member to be inserted in an inside of the sheath, and includes a proximal end movable in a longitudinal direction of the pulling member with respect to the predetermined position in response to a to-and-fro operation on the movement member operation portion; and
a fixation portion that is provided to the first cover, and fixes a distal end of the sheath to the first cover.

2. The distal end cover device according to claim 1,
wherein the sheath can be compressed or expanded with respect to the pulling member.

3. The distal end cover device according to claim 1,
wherein the first cover comprises:
an engagement portion that engages the first cover with the distal end portion; and
a fragile portion configured to be breakable when the first cover is removed from the distal end portion, and
the fixation portion is arranged between the engagement portion and the fragile portion.

4. The distal end cover device according to claim 1,
wherein an entire periphery of the second cover is in close contact with an insulative insulation member and the first cover that are arranged on the outer periphery of the distal end portion.

5. The distal end cover device according to claim 1,
wherein the movement member is a raising base configured to change a movement direction of another insertion device inserted in the insertion portion, in the distal end portion.

6. The distal end cover device according to claim 5,
wherein the coupling member is a shaft portion of the raising base, and
the raising base is swingable centered on the shaft portion.

7. An endoscope, comprising
the distal end portion to which the distal end cover device according to claim 1 is fixed.
